(19) United States
(12) Patent Application Publication
(10) Pub. No.: US 2004/0134084 A1
(43) Pub. Date: Jul. 15, 2004

(54) Method equipment for measuring coverage density of a surface, in particular skin hair coverage

(76) Inventor: Dominique Vanneste, Tournai (BE)

Correspondence Address: James C Wray, Suite 300, 1493 Chain Bridge Road, McLean, VA, 22101, US

(21) Appl. No.: 10/476,796

(22) PCT Filed: Jun. 8, 2001
(86) PCT No.: PCT/EP01/06970

(51) Int. Cl. A61B 1/00

(52) U.S. Cl. 33/512

(57) ABSTRACT

The invention relates to a method for measuring the coverage density of multiple components on a surface, in particular skin hair coverage, by dividing the surface to be measured in a number of zones, comparing the visual appearance of these zones to measuring means comprising reference surfaces assigned with numerical values, so as to allow assigning a numerical value to the measured zones in function of the visual parameter of said series of reference surfaces. The invention also relates to equipment therefor, comprising grid means for dividing the surface to be measured in zones and means for measuring coverage density, involving several sets of density scales comprising n reference surfaces with a visual density which serially decreases by the fractional factors n−1/n, n−2/n, . . . , 1/n.

The invention relates to a method and to equipment for measuring the coverage density (or denseness) of multiple components on a surface, like for instance the coverage of hair on the skin (such as in particular scalp hair density).

The visual evaluation of scalp hair coverage is usually estimated globally by the observers, without instruments. In general one refers to categories of scales to statically classify patterns. When changes are to be monitored over time, this is usually performed on repeat photographs.

Reference is made in this respect to Dawber, R. & Van Neste, D., Hair and Scalp disorders, 1995, Martin Dunitz, London; Kaufman et al., Journal of Investigative Dermatology (JID) 104: p659, 1995; Lilly et al., JID 110: p199, 1998; Kaufman et al., Journal of the American Academy of Dermatology 39, pp. 578-589, 1998; Olsen, A., Dermatologic Therapu 8, pp 18-23, 1998.

By comparing 2 photographs taken at some time difference, a panel of expert readers is rating improvement, worsening or no-change in order to evaluate changes that may have occurred over time, for example in the absence or the presence of a treatment. The information so generated is a composite index reflecting discrete changes of hair follicle function that may be accurately analysed with sophisticated methods.

Reference is made also to U.S. Pat. No. 4,807,163, relating to a computer assisted method for analysing multiple-component fields by creating records with discrete segments of varying intensity, by digitally resolving the intensity of the segments into discrete values, by registering the frequency distribution of said discrete values, and by resolving the frequency distributions through Gaussian analysis.

U.S. Pat. No. 5,331,472 discloses a method and apparatus for counting hair on a defined area using a housing with a magnifying lens and a properly positioned measurement aperture.

The present invention provides a method for, inter alia, global hair evaluation that does not need recording of photographs but only requires a standardised scalp preparation and the use of simple external reference material against which scalp hair coverage can be evaluated comparatively.

The method for measuring the coverage density or denseness of multiple components on a surface, in accordance with this invention, comprises:

dividing the surface to be measured in a number of zones,

comparing the visual appearance of (some of) these zones, which are to be measured, to measuring means comprising a series of reference surfaces having a visual parameter assigned with numerical values, so as to allow assigning a numerical value to the visual appearance of the measured zone(s) in function of the visual parameter of said series of reference surfaces.

According to a preferred embodiment of the invention, applied to skin hair measurement, the method more in particular comprises

dividing the skin surface to be measured in a number of zones,

comparing the visual appearance of (some of) the skin zones, which are to be measured, to measuring means comprising a series of reference surfaces having a visual parameter assigned with numerical values, so as to allow assigning a numerical value to the visual appearance of the measured skin zone(s) in function the visual parameter of said series of reference surfaces.

The measuring means, used in the method of the invention, preferably consist of a transparent support on which said reference surfaces are represented and through which the zone(s) to be measured can be observed.

In preferred embodiments of the invention, the measuring means may in particular comprise at least one set of density scales, showing surfaces having a visual density which decreases with a constant fractional factor, with most preferably at least one reference surface of zero density. This zero density surface can in particular be the entire surface of a transparent support on which the other reference surfaces are represented.

In such embodiments, a zone having the highest coverage density of the surface to be measured, is preferably compared to said set of density scales so as to match its appearance with a surface of the density scales, which is selected as the 100% coverage reference; and the zones to be measured are compared and matched with those surfaces of the density scales which have a visual density less than or equal to said 100% coverage reference, so as to establish a coverage value for said zones, ranging from said 100% value to a 0% value corresponding to a zone without coverage of the surface to be measured, as compared to, respectively observed through, a surface of zero density of the measuring means.

Most preferably, in this embodiment of the invention, the measuring means comprise one first set of density scales comprising n reference surfaces with a visual density which serially decreases by the fractional factors

n−1/n , n−2/n , . . . , 1/n

with respect to reference surface with highest visual density of said first set, and at least one series of n−1 additional sets of density scales, with the visual density of their respective highest density reference surfaces decreasing by the fractional factors

n−1/n , n−2/n , . . . , 1/n

with respect to reference surface with highest visual density of said first set, and with each also n reference surfaces with a visual density which serially decreases by the fractional factors

n−1/n , n−2/n , . . . , 1/n ,

with respect to the visual density of their respective highest density reference surfaces.

More in particular said measuring means may, most appropriately comprise one or more further series of n−1 additional sets of density scales, with the visual density of their respective highest density reference surfaces decreasing by the fractional factors

n−1/n , n−2/n , . . . , 1/n ,

with respect to the reference surface with highest visual density of the n−1^{th} set of a previous series of sets of density scales.

According to still other preferred features of the invention, further means may be used in the method, to provide a reproducible observation position with respect to the object on which coverage density is to be measured.

In the embodiment for measuring scalp hair, these features may in particular involve that for dividing the scalp surface in a number of zones, use is made of transparent grid means through which the scalp is to be observed, and that said means to provide a reproducible positioning comprise reference means intended to be positioned to the head of the object to be measured, and reference marks on said transparent grid means, to be aligned with said reference means during measurement.

Most preferably said transparent grid means (also called “fields grid” or “zoning grid”) may comprise a contour marking to be aligned with the top of a human scalp, and markings subdividing the scalp in a number of zones.

The invention also specifically relates to equipment for measuring the coverage density of multiple components on a surface, comprising grid means for dividing the surface to be measured in zones, and means for measuring coverage density, wherein said means for measuring coverage density comprise

a first set of density scales comprising n reference surfaces with a visual density which serially decreases by the fractional factors

n−1/n , n−2/n , . . . , 1/n

with respect to reference surface with highest visual density of said first set, and

at least one series of n−1 additional sets of density scales, with the visual density of their respective highest density reference surfaces decreasing by the fractional factors

n−1/n , n−2/n , . . . , 1/n

with respect to reference surface with highest visual density of said first set, and with each also n reference surfaces with a visual density which serially decreases by the fractional factors

n−1/n , n−2/n , . . . , 1/n ,

with respect to the visual density of their respective highest density reference surfaces.

More particularly, the measuring means of said equipment preferably comprise one or more further series of n−1 additional sets of density scales, with the visual density of their respective highest density reference surfaces decreasing by the fractional factors

n−1/n , n−2/n , . . . , 1/n ,

with respect to the reference surface with highest visual density of the n−1^{th} set of a previous series of sets of density scales.

The grid means of said equipment may very suitably comprise a transparent surface provided with a circular marking to be aligned with the top of a human scalp, and with grid markings subdividing the scalp in a number of zones.

In accordance with the invention, the equipment may further also comprise means for providing a reproducible measuring position with respect to the object on which coverage density is measured.

These means for providing a reproducible measuring position preferably comprise reference means intended to be positioned to a human head, and further markings provided on said transparent grid means, in a way allowing alignment of said reference means and said further markings on the transparent grid.

The above defined procedure to quantify skin coverage and the equipment (material) to be used, are hereafter described in more detail.

The operating system is composed of a number of external references in order to control critical factors between the observer and the skin. These are named as follows: frontal reference piece, density scales and the fields' grid.

The frontal reference piece is a holder for targets so as to control the distances and angles of observation.

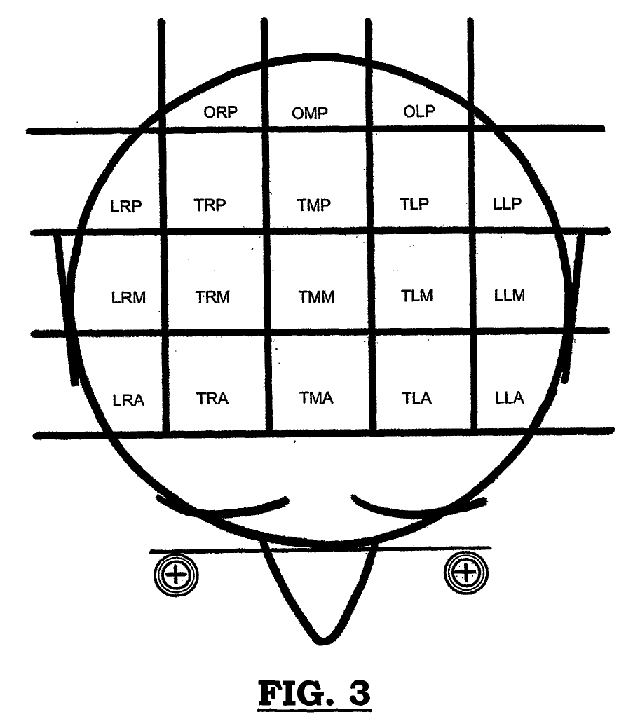

The density scales contain a set of “rulers” or “scales” to characterise the covering density with a set of standard density scales. The fields' grid is a transparent grid for appropriate targeting and topographic definition of the fields under examination. All fields as designed in the grid preferably have the same size; they may appear as squares grouped on a plane with two-dimensions. Any three-dimensional object—including the top of the human head can be projected onto this grid. The names of the fields are given relative to anatomical references of the object. In our example (cf. FIG. 3) the top of the head with its lateral and occipital aspects surrounding the very top, the anterior, mid and posterior segments as well as the left, middle or right part of it, as they are to be evaluated in terms of hair coverage.

The nomenclature letter codes of the scalp zones is explained as follows:

first digit: 0=Occipital, L=Lateral, T=Top;

second digit: L=Left, M=Median, R=Right

third digit: P=Posterior, M=Mid, A=Anterior

Within a set of density scales, the observer then selects a density scale so that the highest density matches appropriately a field representative of completely covered skin (100% coverage) in that subject. By using the lowest density within the selected density scale (which is in fact zero coverage), the observer characterises a totally bare field that may be rated as 0% coverage. Following-up, the observer then rates against the selected density scale each individual field in comparison with the completely covered and uncovered skin field (intermediate density scores ranging from 0% to 100% categorised into 5 classes of density+the common value zero, being the absence of coverage). These measurements characterise hair coverage in a given skin site at a given time. Comparison of measurements made similarly at different times or after some intervention will reflect changes occurring during that time or related to the intervention. The nature of the intervention or the mechanism involved is beyond the scope of the invention. The aim is to measure coverage in a comparative way.

Changes such as natural course of skin or scalp condition, effect of treatment, effect of surgery effect of hair care procedures (styling, shampooing, etc.), can be quantified using the method of the invention.

Materials and Methods—External References—Frontal Reference Piece

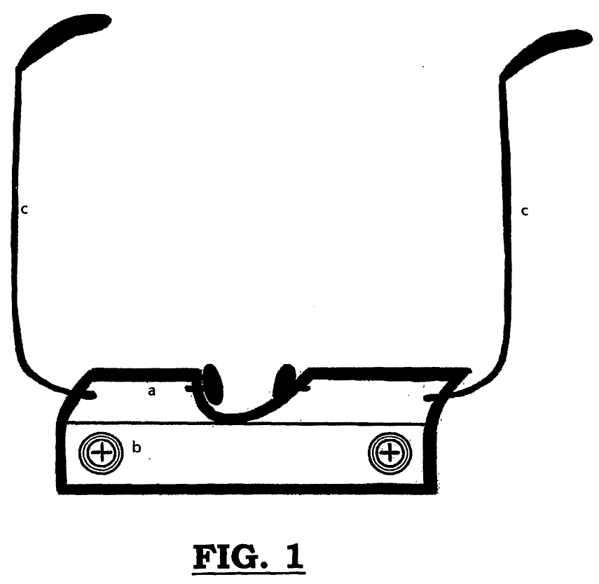

One external reference—named the frontal reference piece—is shown in FIG. 1. This piece involves two planes or faces, for instance at right angle as shown on the figure. A different angle, such as an obtuse angle may also be appropriate or even preferable, depending on the actual measurement circumstances. One face or plane (referred to as (a) in FIG. 1) should be resting on the root of the nose, in contact with the eyebrows, and in continuity with the forehead. The other plane, at a 90° angle, should show at least 2 reference targets (referred to as (b) in FIG. 1) at a pre-set distance. As an example, two branches (or legs or arms) may be used to fix this piece of equipment to the head, such as resting on the junction between ear and scalp (referred to as (c) in FIG. 1).

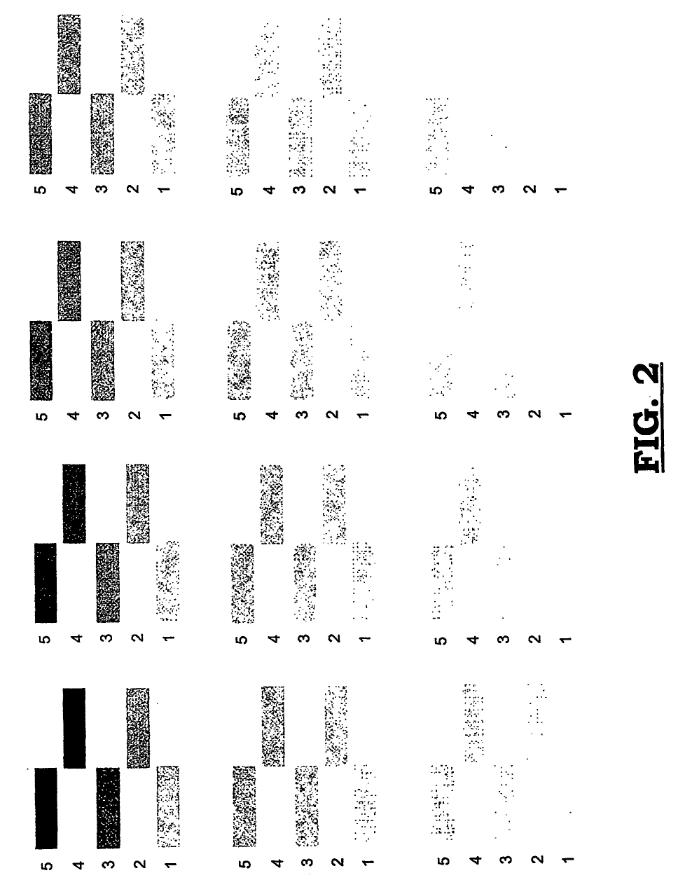

The set of density scales (FIG. 2) is the second external element of the described system.

Each scale can be used separately and shows a range of 6 grades (5 coverage density grades+grade zero) of density ranging between 100% and 0% with a step of 20% (i.e. 80, 60, 40, 20).

In the set of scales, shown in FIG. 2, the 100% coverage value of neighbouring scales decreases serially by 20% from the previous one. As the observer reads rows from left to right and top to bottom the maximum density shifts from 100 to 80, 64, . . . , as illustrated by table I

TABLE I
numerical value matrix of density scales according to FIG. 2

| | | | |
|---|---|---|---|
| 100.00 | 80.00 | 60.00 | 40.00 |
| 80.00 | 64.00 | 48.00 | 32.00 |
| 60.00 | 48.00 | 36.00 | 24.00 |
| 40.00 | 32.00 | 24.00 | 16.00 |
| 20.00 | 16.00 | 12.00 | 8.00 |
| 0.00 | 0.00 | 0.00 | 0.00 |
| 20.00 | 16.00 | 12.00 | 8.00 |
| 16.00 | 12.00 | 9.60 | 6.40 |
| 12.00 | 9.60 | 7.20 | 4.80 |
| 8.00 | 6.40 | 4.80 | 3.20 |
| 4.00 | 3.20 | 2.40 | 1.60 |
| 0.00 | 0.00 | 0.00 | 0.00 |
| 4.00 | 3.20 | 2.40 | 1.60 |
| 3.20 | 2.56 | 1.92 | 1.28 |
| 2.40 | 1.92 | 1.44 | 0.96 |
| 1.60 | 1.28 | 0.96 | 0.64 |
| 0.80 | 0.64 | 0.48 | 0.32 |
| 0.00 | 0.00 | 0.00 | 0.00 |

This series of density scale sets may match even the least or lowest hair density grades as long as the densest zone of the subject almost completely covers the skin behind.

The fields' grid is the 3rd external reference material, as shown in FIG. 3. The grid may be appearing on transparent solid material or preferably be carved out so that the fields are outlined and the solid material does not optically interfere with the skin fields to be examined. The field grid should include the same targets as the ones on the frontal reference piece (as an example see the drawing of FIG. 1b) at an appropriately defined distance. They will serve the purpose of maintaining the appropriate angle and distance between the observer and the fields or zones under examination.

Application example of the method.

The subject is invited to sit in front of the observer on a chair so as to prevent any major movement.

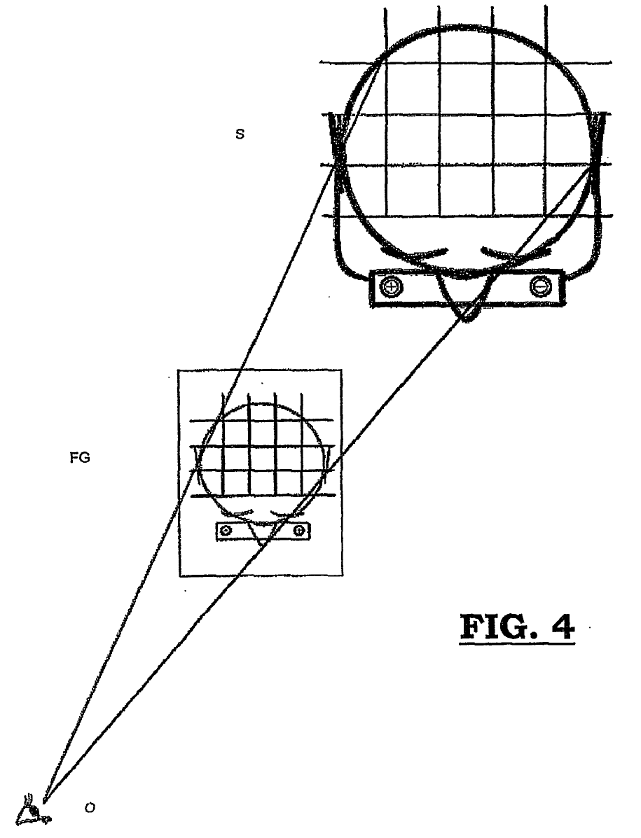

The observer evaluates the matching of the highest grade density scale held in front of the maximally covered field (no skin visible between the hair). When the observer is almost unable to clearly distinguish between density grade 5 and 4 of a given scale as shown in FIG. 2 and the natural hair scalp coverage, the selection of density scale is fixed and will be used later with the other devices (as represented in FIG. 4, showing, schematically, the observer (O), the fields grid (FG) and the subject (S)).

This scale is then moved across the body area to be examined, e.g. the top of the head with the presently developed example, and the natural colour of the skin uncovered by hair is given the lowest density (0% coverage). Each field can be attributed a density grade between 100% and 0% by comparison against the external scales or “rulers”. The density grades shown on this ruler have a typical step of 20%, which is compatible with the purpose of the method. Accordingly grades 1, 2, 3, 4 between no scalp coverage to the highest scalp coverage grade will represent 20,40, 60 and 80% of the maximum skin coverage respectively.

The frontal reference piece is placed on the head of the subject in the recommended position. The subject adopts a standard position so as to show the top of the head to the observer. Matching of the targets shown on the schematic view of the top of the head (FIG. 3) on the 90° external reference (shown in FIG. 1) will allow the observer to place himself at the appropriate distance and vision angle from the subject to be analysed. When the subject has adopted the appropriate position in front of the observer, the targets on the frontal reference piece appear face to face of the observer (FIG. 4) and various fields or zones are observed on the head visible through the grid.

The selected density scale (one of those shown in FIG. 2) may now be applied so as to rate the density in the various fields.

As an example in a case of androgenetic alopecia, one may suggest to start with grades 5 and 4 which will be found essentially at the crown of the head or in the external fields of the grid. The method then suggests looking for a typical bare area. This may be found on the top of the head or on the receding frontal hairline. Usually the bald scalp colour will be rated grade 0. Then the selected density scale can be moved across the various fields under examination. This will allow the observer to evaluate how it matches the density grade of the selected scale and how much hair coverage is present in each field.

Although the size of the fields and the density grade changes have been selected to generate clinically relevant data it may occur that within a given field the density varies somewhat from one corner to the other. It is mentally an easy job to subdivide the field into for instance three rectangles, or four or nine squares which then perfectly match with the hair pattern inside the predefined field. A sum and division of the grades will average the density value ascribed to that field. It is not advisable to subdivide a field into more than nine squares because it is not clinically relevant.

In order to detect potential changes that may occur with time and experience it is advisable to minimise the natural trend to modify a scoring with increasing practice of the method. A reference kit would consist ideally of a number of graded patterns and densities against which the observer can train and control his skill by himself. Another set of blank pictures or schematic representations of affected heads will serve as an internal quality control in order to prevent drifting of the observer rating method. The observer would thus be provided with references which allow to calibrate the scoring system, so as to prevent drift of scoring, and also allow the matching of scoring sessions made at different times.

The method is generally applicable for quantifying proportions of heterogeneous material appearing in a visual field.

Standardised diffuse lighting is advisable but all sorts of light sources and vision angles may be used. Other pieces of equipment may be added whenever desired such as—but not limited to—lenses and specialised light sources, computer recording systems, various ultraviolet, infra-red or other light sources, non optical recording systems, projection of complex or simple patterns on the fields, split light effects, etc.

The method may be used in the skin and hair clinic, at the beauty shop or at the hairdresser's salon, or even at home. Its primary application is to score skin and scalp coverage by hair directly but it may also be used on photographs and computer-stored or web-cam transmitted images, slides, . . .

There is clear evidence that standardisation of scalp hair preparation further improves the experimental reproducibility of the method. The scalp hair should be clean and the hairstyle maintained so as to be comparable at the different evaluation sessions. Natural changes in hair colour which may occur will not, within certain limits, affect substantially the scoring system, but artificial colour and hair dyes, as well as sun-tanning of the scalp should preferably be avoided.

Androgenetic alopecia is the most common condition that affects scalp hair. Therefore it is the condition where the monitoring with this measurement method might be most frequently used. The method may however also be useful in the measurement of other diseases, such as alopecia areata, diffuse hair loss or female pattern hair loss, and various other scalp conditions where genetic or acquired factors such as inflammatory reactions of various origins play a causal role.

The method has a considerable practical value as it can help the observer who is not equipped with sophisticated photographic or computer assisted systems in evaluating the response of a given subject to a specific hair care system or cosmetic or therapeutic modalities. The effect of styling procedures could also be rated and quantified in order to help subjects and hairdressers in the best option for their client when scalp coverage is the evaluation criterion. The quality of hairpieces or wigs could also be rated as well as the quality of scalp graft samples and scalp coverage after scalp hair follicle transplantation.

The method could substantially improve the selection of individuals and defining eligibility before participation in clinical research programmes (drug trial, epidemiological survey, . . . ). It indeed generates more smoothened data as compared to the existing Hamilton-Norwood scales or derived scales. (see articles referred to here above).

### EXAMPLES

Experimental data provide clear evidence that the materials and methods described in the present invention most accurately identify natural changes in androgenetic alopecia, and are able to detect prevention of hair loss and improved hair re-growth.

Known therapeutic regimens for androgenetic alopecia in men, such as the oral intake of Finasteride (actually recommended dosage 1 mg/day) or the application of minoxidil topical solution (actually up to 5%), are typical situations where changes could be detected by this method. The method has also a potential for application when an observer has to discriminate between an active and a non-active substance (such as a placebo) in studies involving quite a small number of individuals (n=10). Hence the system could be used in phase II or pivotal phase III or even during larger phase IV studies in the context of drug finding projects and drug efficacy evaluation. In the dermatology clinic it may help detecting the good responders to a treatment.

In the dermatology clinic there are treatments that may improve 30 to 60% of the patients. It may be of great significance to appropriately detect the good responders to existing and efficacious treatment regimens. If no positive response can be detected after a reasonable period of time in a compliant subject, the present method could help the observer to decide that the subject is not a good responder and to adapt the treatment regimen.

The method and equipment according to the invention can of course be adapted for any kind of observation conditions (such as lighting condition, hairdressing, etc.), and to any kind of manual or automated data recording and data treatment, be it mechanic, electronic, photographic, using information technology systems or not.

1. Method for measuring the coverage density of multiple components on a surface, comprising:
dividing the surface to be measured in a number of zones,
comparing the visual appearance of (some of) these zones, which are to be measured, to measuring means comprising a series of reference surfaces having a visual parameter assigned with numerical values, so as to allow assigning a numerical value to the visual appearance of the measured zone(s) in function of the visual parameter of said series of reference surfaces.

2. Method, according to claim 1, for measuring skin hair coverage, comprising:
dividing the skin surface to be measured in a number of zones,
comparing the visual appearance of (some of) the skin zones, which are to be measured, to measuring means comprising a series of reference surfaces having a visual parameter assigned with numerical values, so as to allow assigning a numerical value to the visual appearance of the measured skin zone(s) in function of the visual parameter of said series of reference surfaces.

3. Method according to anyone of the preceding claims, characterized in that said measuring means consist of a transparent support on which said reference surfaces are represented and through which the zone(s) to be measured can be observed.

4. Method according to anymore of the preceding claims, characterised in that said measuring means comprise at least one reference surface of zero density.

5. Method according to any one of the preceding claims, characterized in that said measuring means comprise at least one set of density scales, showing surfaces having a visual density which decreases with a constant fractional factor;
a zone having the highest coverage density of the surface to be measured is compared to said set of density scales so as to match its appearance with a surface of the density scales, which is selected as the 100% coverage reference; and
the zones to be measured are compared and matched with those surfaces of the density scales which have a visual density less than or equal to said 100% coverage reference, so as to establish a coverage value for said zones, ranging from said 100% value to a 0% value corresponding to a zone without coverage of the surface to be measured, as compared to, respectively observed through, a surface of zero density of the measuring means.

6. Method according to claim 5, characterized in that said measuring means comprise one first set of density scales comprising n reference surfaces with a visual density which serially decreases by the fractional factors
n−1/n , n−2/n , . . . , 1/n
with respect to reference surface with highest visual density of said first set, and
at least one series of n-i additional sets of density scales, with the visual density of their respective highest density reference surfaces decreasing by the fractional factors
n−1/n , n−2/n , . . . , 1/n
with respect to reference surface with highest visual density of said first set, and with each also n reference surfaces with a visual density which serially decreases by the fractional factors
n−1/n , n−2/n , . . . , 1/n ,
with respect to the visual density of their respective highest density reference surfaces.

7. Method according to claim 6, characterised in that said measuring means comprise one or more further series of n−1 additional sets of density scales, with the visual density of their respective highest density reference surfaces decreasing by the fractional factors
n−1/n , n−2/n , . . . , 1/n
with respect to the reference surface with highest visual density of the n−1^{th} set of a previous series of sets of density scales.

8. Method according to any one of the preceding claims, characterised in that further means are used to provide a reproducible observation postition with respect to the object on which coverage density is to be measured.

9. Method according to claim 2 and any one of claims 7 and 8, for measuring scalp hair coverage, characterized in that for dividing the scalp surface in a number of zones use is made of transparent grid means through which the scalp is to be observed, and in that
said means to provide a reproducible positioning comprise reference means intended to be positioned to the head of the object to be measured, and reference marks on said transparent grid means, to be aligned with said reference means during measurement.

10. Method according to claim 9, characterised in that said transparent grid means comprise a circular marking to be aligned with the top of a human scalp, and markings subdividing the scalp in a number of zones.

11. Equipment for measuring the coverage density of multiple components on a surface comprising
grid means for dividing the surface to be measured in zones, and
means for measuring coverage density,
characterized in that said means for measuring coverage density comprise
a first set of density scales comprising n reference surfaces with a visual density which serially decreases by the fractional factors
n−1/n , n−2/n , . . . , 1/n
with respect to reference surface with highest visual density of said first set, and
at least one series of n−1 additional sets of density scales, with the visual density of their respective highest density reference surfaces decreasing by the fractional factors
n−1/n , n−2/n , . . . , 1/n
with respect to reference surface with highest visual density of said first set, and with each also n reference surfaces with a visual density which serially decreases by the fractional factors
n−1/n , n−2/n , . . . , 1/n,
with respect to the visual density of their respective highest density reference surfaces.

12. Equipment according to claim 11, characterised in that said measuring means comprise one or more further series of n−1 additional sets of density scales, with the visual density of their respective highest density reference surfaces decreasing by the fractional factors
n−1/n , n−2/n , . . . , 1/n
with respect to the reference surface with highest visual density of the n−1^{th} set of a previous series of sets of density scales.

13. Equipment according to any one of claim 11 and 12, characterized in that said grid means comprise a transparent surface provided with a circular marking to be aligned with the top of a human scalp, and with grid markings subdividing the scalp in a number of zones.

14. Equipment according to any one of claims 11 to 13, further comprising means for providing a reproducible measuring position with respect to the object on which coverage density is measured.

15. Equipment according to claim 13 and 14, characterised in that said means for providing a reproducible measuring position comprise reference means intended to be positioned to a human head, and further markings provided on said transparent grid means, in a way allowing alignment of said reference means and said further markings on the transparent grid.

16. Kit-of-parts for a method for measuring the coverage density of multiple components on a surface, according to any one of claims 1-10, characterised in that it contains equipment according to any one of claims 11-15.

17. Kit-of-parts for a method according to any one of claims 1-10, characterised in that it contains training means and/or quality control means and/or scoring calibration means, either alone or in addition to equipment according to any one of claims 11-15.
